(19) United States
(12) Patent Application Publication
(10) Pub. No.: US 2021/0128976 A1
(43) Pub. Date: May 6, 2021

(54) RHYTHMIC ATHLETIC TRAINING SYSTEMS AND METHODS

(71) Applicant: Ali C. Barnes, West Chester, OH (US)

(72) Inventor: Ali C. Barnes, West Chester, OH (US)

(21) Appl. No.: 17/149,698

(22) Filed: Jan. 14, 2021

Related U.S. Application Data
(63) Continuation of application No. 15/722,095, filed on Oct. 2, 2017.
(60) Provisional application No. 62/485,117, filed on Apr. 13, 2017.
(60) Provisional application No. 62/402,672, filed on Sep. 30, 2016.

(51) Int. Cl. A63B 23/18; A63B 71/06; A63B 24/00; G09B 19/00; G06Q 30/02; G16H 20/30

(52) U.S. Cl. CPC A63B 23/185; A63B 71/0622; A63B 24/0062; G09B 19/0038; G06Q 30/0255; H04M 1/72403; A63B 2225/685; A63B 2024/0068; A63B 2225/20; A63B 2071/0625; H04M 2250/12; G16H 20/30

(57) ABSTRACT

A portable user device, method and computer program provide rhythmic athletic training assist by tracking an athletic proficiency level of a user. A respective sport control affordance for a plurality of sports are presented on a user interface of the portable user device. In response to a user selection of one sport control affordance associated with a selected sport, more than one drill control affordance for a type of athletic drill associated with the selected sport are presented on the user interface. In response to a user selection of a particular drill control affordance for a particular type of athletic drill, the type of athletic drill is associated with two or more available rhythmic speed settings. Based upon the tracked proficiency level of the user, one of the two or more rhythmic speed settings is selected. Video/audio guide is presented on the user interface at the selected rhythmic speed setting.

### CLAIM OF PRIORITY UNDER 35 U.S.C. § 119

The present Application for Patent is a continuation of U.S. nonprovisional application No. 15/722,095, entitled “RHYTHMIC ATHLETIC TRAINING SYSTEMS AND METHODS” filed Oct. 2, 2017, 2017, and claims priority to U.S. Provisional Application No. 62/402,672, entitled “HI TEKNOLOGY VIDEO MOBILE BEATS ATHLETIC TRAINING APPLICATION” filed Sep. 30, 2016, and to U.S. Provisional Application No. 62/485,117, entitled “RHYTHMIC ATHLETIC TRAINING SYSTEMS AND METHODS” filed Apr. 13, 2017, which are hereby expressly incorporated by reference herein.

### BACKGROUND

### 1. Technical Field

The present disclosure generally relates to an automated presentation of audio visual aids for guiding athletic training, and more particularly to such guidance that is sport specific from more than one selectable sport.

### 2. Description of the Related Art

Becoming a better athlete generally requires intensive training under the supervision of a trainer who pace the training based on a current skill level of a trainee. The trainer needs to have an encyclopedic knowledge of relevant drills and a training schedule that are appropriate for a particular trainee, as well as remembering past performance levels. During the grind of particularly exhausting drills, the trainer can merely exhort or attempt to demonstrate to a degree the skill expected of the trainee. If the trainer is no longer physically capable of demonstrating the drill, then the trainee is left without a clear guide.

It is known that athletes perform with a rhythm that allows them to maximize their skill. When a commentator says that a player is not playing well, they normally use phrases such as “He is out of rhythm”. This statement is made because there is a rhythmic pattern and cadence to most sports. In basketball, the rhythm of the point guard and the pace of the team create the pace of the game. A boxer controls the tempo of the fight with his concentrated rhythm. In football, the footwork of the players is used to disrupt the tempo or timing of the opposing player. In soccer, the speed of the game is played a pace that is controlled by the more aggressive offensive player. The skill level of the person that is most dominant can affect all of these aspects. This skill level can be enhanced by the mobile technology that can be used in an athlete's private time or corporate training. The musical beats enforce learning as our bodies are conditioned to learn through musical beats. Just as a child learns the alphabet through song and stories are learned through nursery rhymes, the body also learns through sounds and repetition. The musical beats gives the user a pattern to duplicate and that pattern can be sped up or slowed to adjust to the skill level of the user for mastery. The video presentation gives a visual of the skill that is to be duplicated. The recording feature allows for the user to self-correct for improved performance. These aspects accent the multiple intelligence theory created by Howard Gardner in his 1983 book Frames of Mind: The Theory of Multiple Intelligences. By enhancing the sensory perception of the user, the better the skill level of the user.

The present invention provides for a novel way to incorporate musical beats as a way to reinforce learning by conditioning to learn through musical beats.

### BRIEF SUMMARY

In one aspect, the present disclosure provides a method including tracking an athletic proficiency level of a user of a portable user device. The method includes presenting, on a user interface, a respective sport control affordance for a plurality of sports. The method includes in response to a user selection of one sport control affordance associated with a selected sport, presenting, on the user interface, more than one drill control affordance for a type of athletic drill associated with the selected sport. The method includes, in response to a user selection of a particular drill control affordance for a particular type of athletic drill, associating the type of athletic drill with two or more available rhythmic speed settings. The method includes selecting one of the two or more rhythmic speed settings based upon the tracked proficiency level of the user. The method includes presenting at least one of a video and an audio guide on the portable user device at the selected rhythmic speed setting.

In another aspect, the present disclosure provides a computer-readable storage medium includes computer-executable instructions, which when executed via a processing unit on a computer performs acts: (i) tracking an athletic proficiency level of a user of a portable user device; (ii) presenting, on a user interface, a respective sport control affordance for a plurality of sports; (iii) in response to a user selection of one sport control affordance associated with a selected sport, presenting, on the user interface, more than one drill control affordance for a type of athletic drill associated with the selected sport; (iv) in response to a user selection of a particular drill control affordance for a particular type of athletic drill, associating the type of athletic drill with two or more available rhythmic speed settings; (v) selecting one of the two or more rhythmic speed settings based upon the tracked proficiency level of the user; and (vi) presenting at least one of a video and an audio guide on the portable user device at the selected rhythmic speed setting.

In an additional aspect, the present disclosure provides a portable user device including a user interface, a memory containing at least one of video and audio guides, and a processor coupled to the user interface and the memory and containing a mobility application that when executed by the processor performs certain acts. An athletic proficiency level of a user of a portable user device is tracked in the memory. A respective sport control affordance for a plurality of sports is presented on the user interface. In response to a user selection of one sport control affordance associated with a selected sport, the user interface presents more than one drill control affordance for a type of athletic drill associated with the selected sport. In response to a user selection of a particular drill control affordance for a particular type of athletic drill, the processor associates the type of athletic drill with two or more available rhythmic speed settings. The processor selects one of the two or more rhythmic speed settings based upon the tracked proficiency level of the user. The user interface of the portable user device presents at least one of a video and an audio guide at the selected rhythmic speed setting.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1 illustrates a block diagram of a communication system including an individually customizable audiovisual editor to obscure objectionable content., according to one or more embodiments;

FIG. 2 illustrates a front view of an electronic device displaying media content that is user customizable to block objectionable content, according to one or more embodiments;

FIG. 3 illustrates a hierarchical menu flow diagram for a mobile application, according to one or more embodiments;

FIG. 4 illustrate a flow diagram of a method of enhancing athletic training through audiovisual guidance, according to one or more embodiments; and

FIG. 5 illustrates a block diagram of example computer-readable medium or computer-readable device including processor-executable instructions configured to embody one or more of the provisions set forth herein, according to one or more embodiments.

### DETAILED DESCRIPTION

The present disclosure provides a portable user device, method and computer program for rhythmic athletic training assist by tracking an athletic proficiency level of a user. A respective sport control affordance for a plurality of sports are presented on a user interface of the portable user device. In response to a user selection of one sport control affordance associated with a selected sport, more than one drill control affordance for a type of athletic drill associated with the selected sport are presented on the user interface. In response to a user selection of a particular drill control affordance for a particular type of athletic drill, the type of athletic drill is associated with two or more available rhythmic speed settings. Based upon the tracked proficiency level of the user, one of the two or more rhythmic speed settings is selected. At least one of a video and an audio guide is presented on the portable user device at the selected rhythmic speed setting.

According to aspects of the present innovation, a mobile application exhibits a philosophy that athletes perform with a rhythm that allows them to maximize their skill. When a commentator says that a player is not playing well, they normally use phrases such as “He is out of rhythm”. This statement is made because there is a rhythmic pattern and cadence to most sports. In basketball, the pace of the game is created by the rhythm of the point guard and the pace of the team. A boxer controls the tempo of the fight with his concentrated rhythm. In football, the footwork of the players is used to disrupt the tempo or timing of the opposing player. In soccer, the speed of the game is played a pace that is controlled by the more aggressive offensive player. All of these aspects can be affected by the skill level of the person that is most dominate. This skill level can be enhanced by the mobile technology that can be used in an athlete's private time or corporate training. The music enforces learning as our bodies are conditioned to learn through music. Just as a child learns the alphabet through song and stories are learned through nursery rhymes, the body also learns through sounds and repetition. The music gives the user a pattern to duplicate and that pattern can be sped up or slowed to adjust to the skill level of the user for mastery. The video presentation gives a visual of the skill that is to be duplicated. The recording feature allows for the user to self-correct for improved performance. These aspects accent the multiple intelligence theory created by Howard Gardner in his 1983 book “Frames of Mind: The Theory of Multiple Intelligences.” By enhancing the sensory perception of the user, the better the skill level of the user becomes.

The mobile application is created in a way that is unique and exclusive when compared to any other application. The overall concept is novel with a range of unlimited rhythm patterns, levels and combinations. The mobile application is constructed to demonstrate athletic movement to specific sound and sophisticated enough to record the individual's movement to grade performance. This application is strictly to enhance the hand, foot, agility, and timing skill of the athlete.

The Mobile Musical Athletic Training Application has multiple levels of performance to achieve the overall skill of athletes. One component is the individual sport aspect. Each sport represented has a sport name along with a musical connotation that will identify the skill that is to be performed. In one embodiment, the names of each skill are titled as follows:
(i) Basketball—“Hip Hop Handles”;
(ii) Football—“Freestyle Footwork”;
(iii) Soccer—“Sure Shot Soccer”; and
(iv) Boxing—“Beat Boxing”.

These aspects can feature a pro-athlete of the chosen sport performing an array of actions that the user has to mimic. The actions performed will be connected with music that has to be matched by the user for mastery. The mobile application can have the ability to change speeds, randomize and customize to the current skill of the user. This makes this application useful for all ages from 4 years of age to adult. The skill level will accommodate novice through professional. The Mobile Musical Athletic Training Application can also stream music and be used as a playlist for standard training.

In one or more embodiments, a mobile application allows the user to be trained by video and audio cadences with instruction from celebrity athlete. The mobile application can use scripted and randomized movement combined with audio recognition to enhance timing. The application can focus on hand, eye, foot, sound and overall movement coordination. Thereby, the mobile application allows the user to train from home versus having to schedule and pay for a personal trainer in the gym. This mobile application promotes individual enhancement through level graduation.

This application can have an advanced stage which includes but not limited to: (i) Recording yourself (video, Sound), (ii) Augmented Reality, and (iii) complimentary devices that connect via blue tooth such as (Watch or bracelet, accelerometer, headphones etc.). This application can include video, sound, music, commentary and photos of celebrity athletes. The mobile application can consist of 4 or more different segments, such as football, basketball, boxing and soccer. The mobile application can serve a wide demographic, children through professional level. The application can have video and music that can be sped up, slowed down, customized and randomized to increase skill and perfection. The application will have a grading component that rates the users' performance. Application can have a playback function to show the user their performance.

The mobile application can show video of specific celebrity athlete of specific sport performing drills and training aspects of that sport. The mobile application can have a landing page (Home Page). The mobile application can have a selection page (Football, Basketball, Soccer and Boxing). The mobile application can have a music only option. This music may be streamed and downloaded. This function allows for generic non-sport specific training with music that serves as motivation for the user. Videos can accompany the application as a useful aspect of the learning and functionality component. In wearable technology, the application records movement and timing to give data based feedback that the user can analyze performance. In the standard mobile version the user will be able to watch the athlete, hear the music and work to imitate the instructions given.

Analytics are tracked from the mobile application for coaching and training instructions. The user will have detailed data to help them train at a higher level and increase their pace. There is no other mobile application that will allow a user to have exclusive music, recording capabilities for functional competency and unlimited training levels associated with the ability to enhance the important aspects of motor functioning. All of these aspects combine for a legitimate sports application.

TABLE A provides related aspects of the present innovation:

TABLE A

| First Attribute | Second Related Attribute | Third Related Attribute |
|---|---|---|
| Train for Sports by Music | Movement to Music is | Each Definitive Movement Done |
| Application | Graded for Accuracy and Pace | is on the Metronome of Music |
| The Pace of Music Mimics | Workouts Can Increase | Workouts Can Be Customized |
| the Pace of Movement | and Decrease in Pace | and Randomized By User |
| Augmented Reality Places | Virtual Reality Aspects | Training Can Be Individual |
| User Next to Selected Athlete | Connecting Athlete with | Conducted Anywhere |
| During Training | User | |
| Training Can Be Conducted As | App Can Be Used As a | App Can Be Used As a |
| a Group in a Session | Showcase For Athletic | Showcase For Athletic |
| | Statistics | Statistics |
| More Workouts Can Be | App Can Be Shared on All | Workouts Can Be Saved |
| Purchased as In App Upgrades | Social Media Instantly | and Categorized |
| Proprietary and Unique Songs | Compatible with Wearable | |
| Can Be Streamed Without | Technology Such As | |
| Connecting to Workouts | Watches, Sleeves, | |
| | Wristbands, etc. | |

In the following detailed description of exemplary embodiments of the disclosure, specific exemplary embodiments in which the disclosure may be practiced are described in sufficient detail to enable those skilled in the art to practice the disclosed embodiments. For example, specific details such as specific method orders, structures, elements, and connections have been presented herein. However, it is to be understood that the specific details presented need not be utilized to practice embodiments of the present disclosure. It is also to be understood that other embodiments may be utilized and that logical, architectural, programmatic, mechanical, electrical and other changes may be made without departing from general scope of the disclosure. The following detailed description is, therefore, not to be taken in a limiting sense, and the scope of the present disclosure is defined by the appended claims and equivalents thereof.

References within the specification to “one embodiment,” “an embodiment,” “embodiments”, or “one or more embodiments” are intended to indicate that a particular feature, structure, or characteristic described in connection with the embodiment is included in at least one embodiment of the present disclosure. The appearance of such phrases in various places within the specification are not necessarily all referring to the same embodiment, nor are separate or alternative embodiments mutually exclusive of other embodiments. Further, various features are described which may be exhibited by some embodiments and not by others. Similarly, various requirements are described which may be requirements for some embodiments but not other embodiments.

It is understood that the use of specific component, device and/or parameter names and/or corresponding acronyms thereof, such as those of the executing utility, logic, and/or firmware described herein, are for example only and not meant to imply any limitations on the described embodiments. The embodiments may thus be described with different nomenclature and/or terminology utilized to describe the components, devices, parameters, methods and/or functions herein, without limitation. References to any specific protocol or proprietary name in describing one or more elements, features or concepts of the embodiments are provided solely as examples of one implementation, and such references do not limit the extension of the claimed embodiments to embodiments in which different element, feature, protocol, or concept names are utilized. Thus, each term utilized herein is to be given its broadest interpretation given the context in which that terms is utilized.

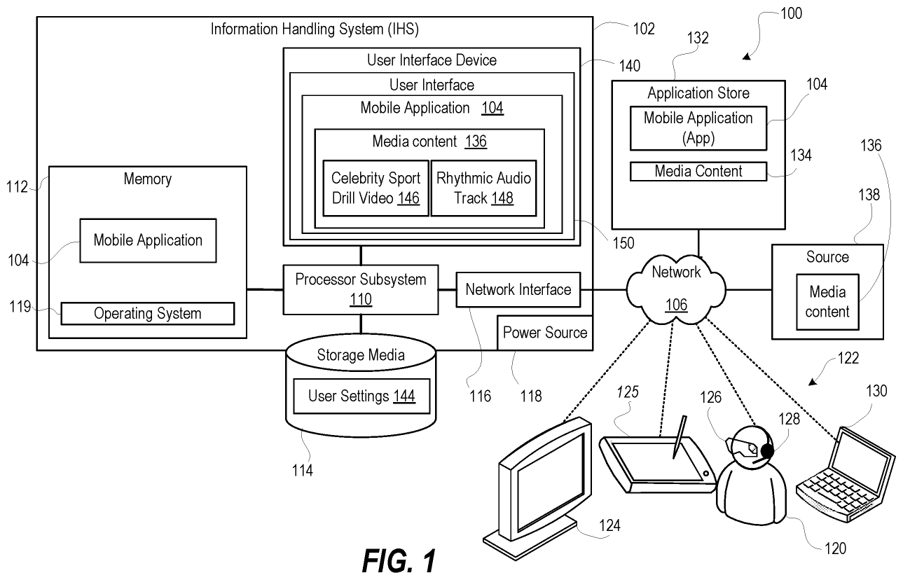

FIG. 1 illustrates a communication system 100 including an information handling system (IHS) 102 for executing a mobile application 104 for rhythmic and enhanced athletic training at least in part over a network 106. For purposes of this disclosure, an information handling system, such as IHS 102, may include any instrumentality or aggregate of instrumentalities operable to compute, classify, process, transmit, receive, retrieve, originate, switch, store, display, manifest, detect, record, reproduce, handle, or utilize any form of information, intelligence, or data for business, scientific, control, or other purposes. For example, an information handling system may be a handheld device, personal computer, a server, a network storage device, or any other suitable device and may vary in size, shape, performance, functionality, and price. The information handling system may include random access memory (RAM), one or more processing resources such as a central processing unit (CPU) or hardware or software control logic, ROM, and/or other types of nonvolatile memory. Additional components of the information handling system may include one or more disk drives, one or more network ports for communicating with external devices as well as various input and output (I/O) devices, such as a keyboard, a mouse, and a video display. The information handling system may also include one or more buses operable to transmit communications between the various hardware components.

In a particular embodiment, the IHS 102 includes a processor subsystem 110, a memory 112 communicatively coupled to processor subsystem 110, storage media 114, a network interface 116 communicatively coupled to processor subsystem 110, and a power source 118 electrically coupled to processor subsystem 110. Processor subsystem 110 may include any system, device, or apparatus configured to interpret and/or execute program instructions and/or process data, and may include, without limitation a microprocessor, microcontroller, digital signal processor (DSP), Application Specific Integrated Circuit (ASIC), or any other digital or analog circuitry configured to interpret and/or execute program instructions and/or process data such as an operating system 119. In some embodiments, processor subsystem 110 may interpret and/or execute program instructions and/or process data stored in memory 112 and/or another component of IHS 102. Memory 112 may be communicatively coupled to processor subsystem 110 and may include any system, device, or apparatus configured to retain program instructions and/or data for a period of time (e.g., computer-readable media). By way of example without limitation, memory 112 may include RAM, EEPROM, a PCMCIA card, flash memory, magnetic storage, opto-magnetic storage, or any suitable selection and/or array of volatile or non-volatile memory that retains data after power to IHS 102 is turned off or power to IHS 102 is removed. Network interface 116 may include any suitable system, apparatus, or device operable to serve as an interface between IHS 102 and network 106. Network interface 116 may enable the IHS 102 to communicate over network 106 using any suitable transmission protocol and/or standard, including without limitation all transmission protocols and/or standards enumerated herein with respect to the discussion of network 106.

The processor subsystem 110 of the IHS 102 or electronic device 122 selects media content 136 for presentation on a user interface 150 of user interface device 140. The selected media content 136 contains a selected at least one of: (i) words and (ii) images. The processor subsystem 110 associates a user 120 of the electronic device 122 with a selected one or more objectionable items. The processor subsystem 110 identifies at least one instance of a selected objectionable item; and (d) obscures presentation of the selected objectionable item during presentation of the selected media content without altering any recorded version of the selected media content.

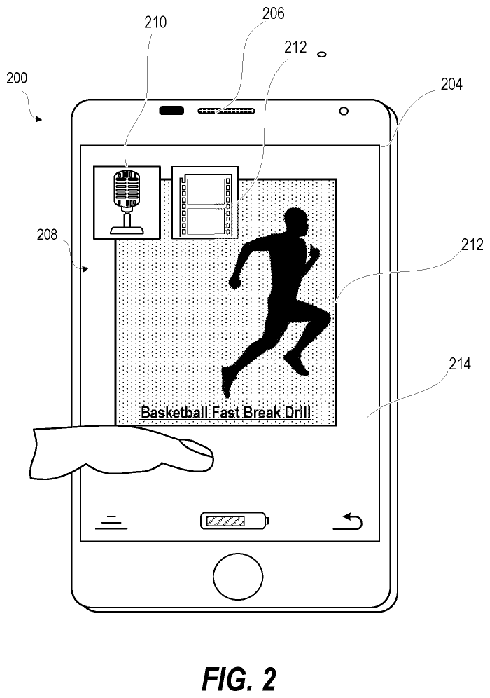

FIG. 2 illustrates an illustrative electronic device 200 having a user interface device 140 (FIG. 1) that includes a visual display 204 and a speaker 206. Affordances 208 can be presented such as visually or aurally to solicit user inputs. For example, a music icon 210 can activate streaming audio content. A video icon 212 can launch a celebrity sport training video of a mobile application 214.

IHS 102 may further allow partner and third-party entities to user interface with IHS 102 to provide additional products or services that leverage and/or interoperate with the features offered by IHS 102 through user interfaces. For example, a partner or third party entity may produce sensors or wearable athletic performance monitoring devices that are compatible with the services offered by IHS 102. In particular, the wearable monitoring devices or sensors may integrate one or more features or services provided by IHS 102 such as viewing the user's workout information in conjunction with a competitor's workout information (e.g., competitor workout information may be retrieved remotely from IHS 102 through the wearable device or sensor). Compatibility may include operation under secure protocols understood and/or required by IHS 102. In another example, athletic activity equipment such as tennis balls, basketballs, tennis rackets, lacrosse sticks, gold clubs, golf balls, soccer balls, baseballs, football pads and the like may be produced by third party or partner entities with technology that allows interfacing with IHS 102. For example, tennis balls may include sensors to detect a force of a user's stroke. Such information may be transmitted from the tennis ball to another device or to IHS 102 for analysis, storage and/or monitoring. In another example, football pads may include pressure sensors to indicate an amount of force with which football players are being hit or that the players are exerting.

IHS 102 may, in one or more arrangements, include a marketing engine that leverages activity information collected from users to identify consumer interests, activity patterns and trends. This information may be used to determine what information to display to users and what products or services to advertise. For example, if a user frequently plays basketball and purchases basketball related equipment, a service may be able to better target the user with advertisements that relate to basketball events, sales and products. In another example, if a user frequently tracks calories (e.g., as opposed to miles or pace), a service may target the user with weight loss services and/or products.

IHS 102 may use various analytical engines that are configured to process the submitted athletic activity information (or other types of information such as shopping behavior and browsing trends) to customize the user experience. In one example, a system may include a consumer-profiling engine configured to customize content based on a user interests and other aspects of a user's profile. For example, the system may collect eCommerce data, event participation data, workout information, music selection information and the like to produce insights into various aspects of the user. Such insights may include user interests in sports, personal styles and strengths, user preferences for events or event locations, shopping and/or browsing interests, community participation (e.g., social networking membership) and the like. Based on these determined insights, a system such as IHS 102 may customize various aspects of a user's experience using IHS 102 including suggesting various products (or a color scheme thereof), services, events and the like for the user. For example, a user's homepage on an athletic performance monitoring site may be customized with information including articles, advertisements, messages and the like that is determined to be of particular interest to the user. In another example, information may be prioritized for display to the user based on the user profile. According to one arrangement, metadata may be stored in association with products and used to compare and match against keywords corresponding to interests, insights or preferences determined from and associated with a user. Such matching and customization may also be performed on a group level, for example, based on demographics, location, preferred sport, and the like. Accordingly, products, events and services may be identified for a particular category or group of people based on shared interests or characteristics by matching those interests or characteristics to the determined insights.

IHS 102 may further include a consumer behavior engine. For example, a consumer behavior engine may be used to collect information about user behavior and to use such behavior to optimize consumer services offered to the user, customize a navigation flow of various interactive sites and services and to increase a customer behavior knowledge database. In one example, IHS 102 may modify site content or navigation flows if consumer behavior engine identifies that a user frequently uses a particular functionality that is not displayed on a main page. That is, IHS 102 may customize the user's main page by adding the frequently and/or recently used functionality. Additionally or alternatively, a produce or service provider may customize their product line or manufacturing capabilities based on the results of the consumer behavior engine. This may help a service or product provider meet demands and maximize profits. Further, in some arrangements, products, services or events may be showcased at various venues such as retail stores based on the consumer behavior analysis. In one example, an electronic bulletin board of a retail store may display advertisements based on what has been determined to be popular based on the collected user behavior information. In other arrangements, the electronic bulletin board may update its display on an individual basis. The individual may wear or carry some electronic identifier that may be detected by the bulletin board. Upon detecting the individual, the bulletin board may retrieve preference or characteristic information for the user to identify products, services and/or events to advertise on the board.

In one or more arrangements, IHS 102 may also include an event services engine that is configured to create events based on information collected from users, their behavior and experiences. For example, an example event services engine may collect various types of information including event registration information, athletic performance metrics, athletic performance histories, fundraising information, event related purchase histories and the like to evaluate the effectiveness and draw of past events. Based on the successes and failures thereof (identified through the analysis of the collected information), event services engine may create events that specifically target various types of audiences and participants or to achieve a specified objective. In one example, event services engine may create events that are designed to maximize fundraising. In yet another example, event services engine may create events that seek to maximize attendance.

Using the above analytical engines, a user's experience with a fitness-oriented service or system (IHS 102) may be improved. In particular, users that are not inclined to exercise may be more willing or motivated to access the fitness-oriented service or system since the service pages or user interfaces may be customized to the user's particular interests. In some examples, the customization of a user's experience may include identifying content that is related to a non-athletic activity interest of the user (e.g., entertainment news, music, movies, etc.).

In one or more embodiments, the information collected using a system such as IHS 102 may be used to provide various ways to motivate and encourage athletic activity among users. According to one or more aspects, measures of athletic activity may be converted into a form of currency that may be used to purchase products, measure an overall athletic activity level, receive rewards, achieve goals, obtain discounts, use in video games or other applications and the like. For example, calories burned during a running workout may be converted into activity points based on a calorie-to-activity point conversion rate. In another example, activity points may be rewarded for running activities based on a distance run rather than or in addition to the number of calories burned. In another example, a number of steps taken during a walking workout may be converted into athletic activity points based on an activity point conversion rate. In yet another example, activity points may be calculated for a weightlifting exercise based on the amount of weight lifted and a number of repetitions performed using that weight. As other examples, time involved at a predetermined exertion level and/or improving on a personal best may result in activity point awards.

Different conversion factors, rates and algorithms may be applied to different types of athletic activity. In one or more arrangements, a conversion rate or algorithm may be selected based on user characteristics such as weight, gender, height, age, resting heart rate, an activity level and/or combinations thereof. Conversion rates may also be stored and implemented within various types of gym equipment. Accordingly, a gym device may calculate a number of activity points earned and transmit that information to the user's monitoring device. Alternatively or additionally, the gym device may provide the conversion rate and the athletic measures to the monitoring device, thereby allowing the monitoring device to perform its own calculation/conversion. In some instances, if a user fails to achieve a goal or reach an objective, points may be deducted from the user.

According to one or more additional aspects, a conversion rate or algorithm may be determined based on a user's athleticism score such as a SPARQ rating. Alternatively or additionally, data used in deriving the SPARQ rating may be evaluated in determining a number of points to award or deduct from a user.

Athletic activity, as described herein, may be monitored and tracked using an athletic activity performance-monitoring device such as performance monitoring device 200 of FIG. 2. Once athletic activity has been recorded by monitoring device 200, device 200 may convert the recorded athletic activity into activity points. For example, upon completion of the workout, a user may select an option to display the number of activity points earned through the workout. Alternatively or additionally, the athletic activity may be converted into activity points on-the-fly. For example, as mileage or steps are accumulated or as calories are burned during a workout, the athletic activity performance monitoring device 200 may determine an equivalent number of activity points. These activity points may be displayed on a display, e.g., a mobile device, so that a user may be provided with additional motivation to keep working out. That is, since athletic activity points may be used as a form of currency, a user may be more motivated to continue working out to be able to purchase a product or service that costs a certain number of activity points sooner.

In some arrangements, activity points may also be earned through activities other than athletic activities. For example, user purchases, visiting advertiser's websites, signing up for or participating in an event and the like may earn a user activity points. Points may also be deducted from a user for various activities. For example, the types of food a user eats may affect the user's point balance. Accordingly, in one particular example, if a user eats something considered unhealthy such as a donut, a certain number of activity points may be deducted form the user's account. The deduction may correspond, in one or more arrangements, to a number of calories associated with that food product. In contrast, if a user consumes food that is considered to be healthy and/or nutritious, the user may earn points based on that activity as well. Thus, in one specific example, a user may consume two servings of vegetables. Accordingly, the user may earn a certain number of points for consuming those two servings of vegetables.

Athletic performance monitoring devices may be configured specifically for one or more types of athletic activity or may provide generic tracking and monitoring for a variety of different athletic activity types. In the latter instance, one example of a generic performance monitoring device may be one that converts all movement to a number of steps taken (e.g., using a pedometer). Accordingly, regardless of whether the user is running, playing soccer, playing tennis or jumping rope, the athletic activity measure may register in steps. Accordingly, the number of athletic activity points earned through the use of such a generic monitoring device may use a step-based conversion rate. In another example, an athletic performance monitoring device may be configured to detect measures of athletic activity that are specific to a particular sport or athletic activity. For example, a monitoring device specifically configured for running may include sensors and/or software that provide running pace detection at which a user is running as well as a distance run. The running specific monitoring device may further be configured to use pace and distance information in determining a number of activity points corresponding to a running workout. In another example, a monitoring device may be configured to monitor and track soccer-related activities. Accordingly, the monitoring device may include sensors for detecting contact between a user and a ball and/or a force of impact between a user's foot and the ball for example. A conversion algorithm may then be used that incorporates such soccer-specific information in determining a number of activity points earned in a soccer workout. Other types of sports of athletic activities such as basketball, football, cycling, using gym equipment and the like may also have activity-specific measures. In addition to activity specific components and/or detection algorithms, athletic performance monitoring devices may also include generic detection systems so that the devices may be used in a wide variety of activities. For example, each of the above soccer athletic performance monitoring device and running performance monitoring device may include a general pedometer for detecting activity during non-soccer and non-running type activities, respectively.

Devices might also be configured to be sport-specific by defining a subset of athletic activity measures that may be monitored. For example, in a football configuration, the device may be configured to only measure heart rate and pace. In another example where the monitoring device is configured for soccer, the device might be configured to only monitor speed and football impact force. Additionally or alternatively, the type and content of feedback including motivational messages and coaching provided to the user may be sport-specific or selected based on the sport for which the monitoring device is configured. Accordingly, different messages may be provided to users depending on the sport for which the device is configured.

Moreover, by configuring a device for a particular sport, a trainer or coach may be able to push information to users based on sport. Accordingly, a trainer may develop or provide workouts that are intended for athletes in a particular sport. In such instances, the trainer may transmit the workout data to only those devices that are configured for the relevant sport or sports. In another example, a coach may transmit training profiles to each of the members of a team for a specific sport. Similarly, challenges, goals and other objectives may also be defined and transmitted in a sport-specific manner. In some arrangements, a device configured for a particular sport may ignore or discard data that is not intended for the particular sport.

Alternatively or additionally, devices may be classified into multiple categories such as certified and non-certified devices. These classifications may be defined by an entity or organization that provides user athletic activity performance monitoring service and/or that sponsors activity point rewards. The classifications may further affect the amount or grade of activity points earned. For example, data recorded by certified devices may be treated differently from data recorded by non-certified devices. As a more specific example, activity points earned through certified devices may be considered more valuable or reliable than activity points earned through manual entry or non-certified devices.

In one or more arrangements, software, algorithms, product labels and/or services may be licensed to other companies for the creation of certified devices, software and/or hardware. Accordingly, third party companies or developers may create applications or add-on hardware that utilizes the activity performance monitoring features provided by an athletic activity performance monitoring and tracking service. For example, a third party developer may use application protocol user interfaces (APIs) of a device to access activity tracking capabilities and information. The information may then be incorporated into third party software, such as a video game in which progression is measured by the user's athletic performance. In another example, a developer may create a mobile application that helps select music based on a current pace at which a user is running or walking, a number of steps taken, a number of calories burned, a rate at which a user is burning calories and the like. Accordingly, the mobile application may use device or application specific user interfaces to access the necessary athletic activity information. The APIs and/or other tools that may be used by third party developers may include security mechanism to prevent unauthorized access to athletic activity information.

Alternatively or additionally, athletic performance data including, for example, miles run, calories burned, activity points earned and the like, may be transmitted between athletic performance monitoring devices such as device 200 and/or a remote athletic monitoring system. The transfer of data between devices may be performed through wired or wireless connections and networks including infrared connections, BLUETOOTH connections, wired and wireless local area networks, cellular networks, wide area networks such as the Internet and the like. The transmission of athletic information including activity points among performance monitoring devices, athletic activity data monitoring and tracking systems, non-athletic activity monitoring devices and other systems may be encrypted and/or transmitted using a secure or proprietary protocol to prevent unauthorized interception, access and/or tampering of such data. For example, Pretty Good Privacy (PGP) encryption may be used to encrypt athletic activity information. In another example, data may be transmitted using Secure Socket Layer (SSL) protocol to insure security. If the data is not encrypted according to an agreed-upon encryption algorithm or transmitted using a specified transmission protocol, the data may be rejected or communications in general may be denied.

Transmission and sharing of athletic activity information including activity points may allow a user to use multiple different athletic activity performance monitoring devices and/or other devices (e.g., personal computers, netbooks, mobile phones). For example, if a user uses different types of monitoring devices for different types of athletic activity, the user may wish to track an overall athletic activity level across all workouts and devices. Accordingly, the information collected and/or determined through each of the multiple devices may be shared and synchronized. However, in some configurations, activity point synchronization may require authorization through an activity point service to insure that activity points are not falsified, duplicated or modified in an unauthorized manner. For example, a user may be required to upload activity data to an activity point service from a first device and subsequently synchronize that data to each of one or more other devices the user owns through the activity point service. The activity point service may insure the legitimacy of synchronization between devices using such a configuration.

In addition to athletic activities, a mobile device such as a performance monitoring device may monitor and track other types of activities such as a user's reading habits, shopping trends, browsing behavior, gaming interests, event participation history and the like. For example, mobile devices may include network communication capabilities that allow users to access a network such as the Internet. Accordingly, a user may use a mobile device not only to track and store athletic activity data but also to conduct on-line shopping, read articles, sign-up for events, play games, chat with other users and the like. The mobile device may thus be configured to record a history of the user's behavior such as shopping history, browsing history, gaming history, friends list, event participation history and the like.

Data for other users might also be collected for a current user. For example, if a user wishes to track the athletic performance of a friend, the user may use his or her monitoring device to download or receive data for the friend. Such a process may require approval by the other user. This information, e.g., the type of information of the friend that the user views may also be used in determining user preferences, recommendations, suggestion; and/or customizing user experiences.

Additional information may also be gleaned from the collected data including a health status of a user. For example, heart conditions, high or low blood pressure, stress levels, overall physical condition and the like may be determined based on heart rate measurements, pace information, calories burned, distance run and the like. Additionally or alternatively, one or more portions of the collected data may be used to determine an overall athleticism score (e.g., SPARQ score).

A user 120 can use one or more electronic devices 122, such as a television monitor 124, a tablet or smartphone device 125, augmented or virtual reality goggles 126, headphones 128 and personal computing workstation 130. Each can perform some or all of the processing as the IHS 102. In some instances, a remote IHS 102 performs distributed processing. Each of the electronic devices 122 and IHS 102 can be provisioned with the mobile application 104 or download a copy from an application store 132. The application store 132 can also maintain a network repository 134 that contains a database of identified users associated with a training history and subscription. Thereby, user devices 122 can be enabled with previously customized user settings 144 for a particular user. Electronic devices 122 can also be provisioned with media content 136 or download media content 136 from a source 138 over the network 106. Examples of media content include celebrity videos 146 and audio tracks 148 tailored for a particular type of sport or drill. Alternatively or in addition, customization as user settings 144 can be locally stored in storage media 114.

The processor subsystem 110 of the IHS 102 or electronic device 122 selects media content 136 for presentation on a user interface device 140. The selected media content 136 contains a selected at least one of: (i) words and (ii) images. The processor subsystem 110 associates a user 120 of the electronic device 122 with a selected one or more objectionable items.

The processor subsystem 110 identifies at least one instance of a selected objectionable item; and (d) obscures presentation of the selected objectionable item during presentation of the selected media content without altering any recorded version of the selected media content.

FIG. 2 illustrates an illustrative rhythmic athletic training assist electronic device 200 having a user interface device 202 that includes a visual display 204 and a speaker 206. Affordances 208 can be presented such as visually or aurally to solicit user inputs. For example, a music icon 210 can activate streaming audio content. A video icon 212 can launch a celebrity sport training video of a mobile application 214.

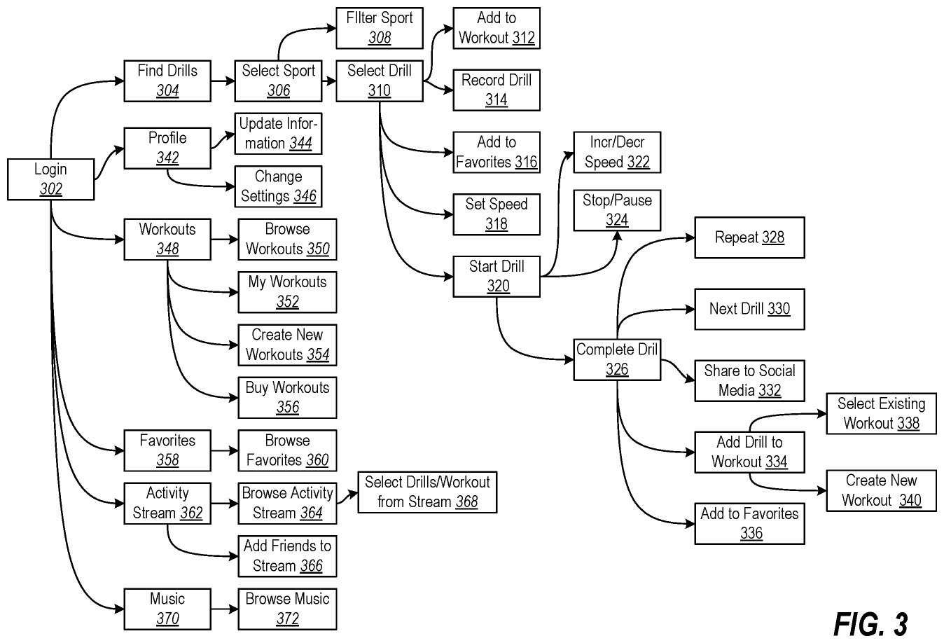

FIG. 3 illustrates a graphical user interface (GUI) command hierarchy 300 for interacting with the mobile application 214 (FIG. 2). The command hierarchy 300 can be entered into via a login component (block 302) that has a find drills selection (block 304) from which one or more select sport affordances (block 306) can be made. For example, a filter sport affordance (block 308) can assist the user in finding a sport of interest. Once a sport is found, a select drill affordance (block 310) allows the user to customize an athletic drill to be performed with the assistance of the mobile application. For example, a user interface can provide the following options: add to workout affordance (block 312), record drill affordance (block 314), add to favorites affordance (block 316), set speed affordance (block 318), and start drill affordance (block 320). Upon selecting the latter, additional control affordances can be presented such as increment/decrement speed affordance (block 322), stop/pause drill affordance (block 324), and complete drill affordance (block 326). Selection of the latter can open addition selections such as: (i) repeat affordance (block 328), (ii) next drill affordance (block 330), (i) share to social media affordance (block 332), (i) add drill to workout affordance (block 334), and (i) add to favorites affordance (block 336). Selection of the add to drill affordance (block 334) can enable selection of either a select existing work affordance (block 338) or create new workout affordance (block 340).

Returning to login affordance (block 302), another option linked thereto can be a profile affordance (block 342) from which a user can select either an update information affordance (block 344) or a change settings affordance (block 346). Returning to login affordance (block 302), another option linked thereto can be a workouts affordance (block 348) from which a user can select one of: (i) a browse workouts affordance (block 350), (ii) my workouts affordance (block 352), (iii) a create new workouts affordance (block 354), and (iv) buy workouts affordance (block 356). Returning to login affordance (block 302), another option linked thereto can be a favorites affordance (block 358) from which a user can select a browse favorites affordance (block 360).

Returning to login affordance (block 302), another option linked thereto can be an activity stream affordance (block 362) from which a user can select either a browse activity affordance (block 364) or an add friends to stream affordance (block 366). From the former, the user can select drills/workout from stream affordance (block 368). Returning to login affordance (block 302), another option linked thereto can be a music affordance (block 370) from which a user can select a browse music affordance (block 372).

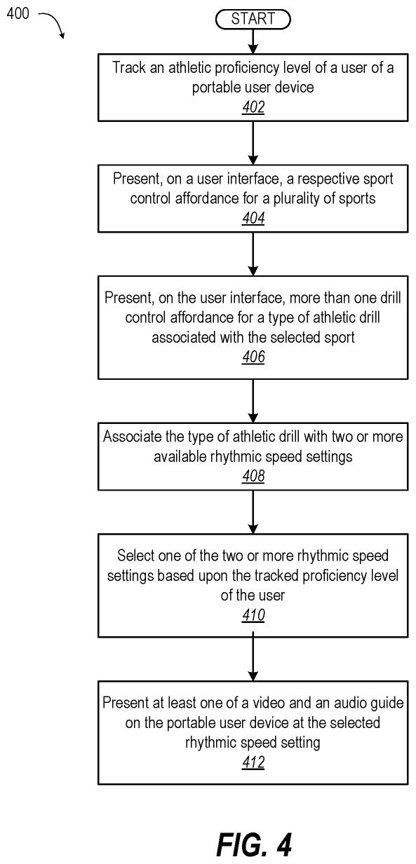

FIG. 4 illustrates a method 400 for rhythmic athletic training of a user of a portable user device. In one or more embodiments, method 400 includes tracking an athletic proficiency level of a user of a portable user device (block 402). Method 400 includes presenting, on a user interface, a respective sport control affordance for a plurality of sports (block 404). Method 400 includes, in response to a user selection of one sport control affordance associated with a selected sport, presenting, on the user interface, more than one drill control affordance for a type of athletic drill associated with the selected sport (block 406). Method 400 includes, in response to a user selection of a particular drill control affordance for a particular type of athletic drill, associating the type of athletic drill with two or more available rhythmic speed settings (block 408). Method 400 includes selecting one of the two or more rhythmic speed settings based upon the tracked proficiency level of the user (block 410). Method 400 includes presenting at least one of a video and an audio guide on the portable user device at the selected rhythmic speed setting (block 412).

In one or more embodiments, method 400 includes presenting a rhythmic speed adjustment affordance on the user interface; and in response to receiving a user selection of the rhythmic speed adjustment affordance, changing the presented at least one of the video and the audio guide to conform with an adjusted rhythmic speed setting.

In one or more embodiments, method 400 includes creating a workout comprising more than one athletic drill; modifying at least one drill in response to a user selection to create a modified workout; and tracking performance of the modified workout for the user.

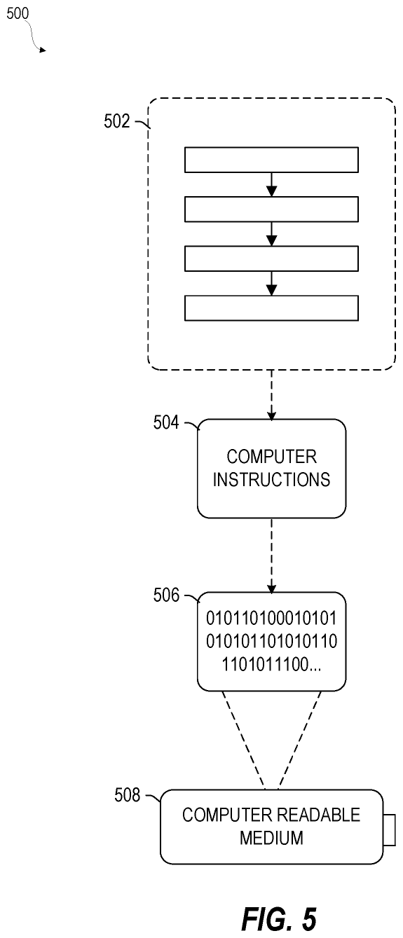

Still another embodiment involves a computer-readable medium including processor-executable instructions configured to implement one or more embodiments of the techniques presented herein. An embodiment of a computer-readable medium or a computer-readable device devised in these ways is illustrated in FIG. 5, wherein an implementation 500 includes a computer-readable medium 508, such as a CD-R, DVD-R, flash drive, a platter of a hard disk drive, etc., on which is encoded computer-readable data 506. This computer-readable data 506, such as binary data including a plurality of zero's and one's as shown in 506, in turn includes a set of computer instructions 504 configured to operate according to one or more of the principles set forth herein. In one such embodiment 500, the processor-executable computer instructions 504 may be configured to perform a method 502, such as method 400 of FIG. 4. In another embodiment, the processor-executable instructions 504 may be configured to implement a system, such as the system 100 of FIG. 1. Many such computer-readable media may be devised by those of ordinary skill in the art that are configured to operate in accordance with the techniques presented herein.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure not be limited to the particular embodiments disclosed for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

What is claimed is:

1. A method comprising:
tracking an athletic proficiency level of a user of a portable user device;
presenting, on a user interface, a respective sport control affordance for a plurality of sports;
in response to a user selection of one sport control affordance associated with a selected sport,
presenting, on the user interface, more than one drill control affordance for a type of athletic drill associated with the selected sport;
in response to a user selection of a particular drill control affordance for a particular type of athletic drill, associating the type of athletic drill with two or more available rhythmic speed settings;
selecting one of the two or more rhythmic speed settings based upon the tracked proficiency level of the user; and
presenting a video depicting one or more of scripted and randomized movement and an audio guide comprising an audio cadence on the portable user device both at the selected rhythmic speed setting that are combined using audio recognition to enhance timing.

2. The method of claim 1, further comprising:
presenting a rhythmic speed adjustment affordance on the user interface;
in response to receiving a user selection of the rhythmic speed adjustment affordance, changing the presented at least one of the video and the audio guide to conform with an adjusted rhythmic speed setting.

3. The method of claim 1, further comprising:
creating a workout comprising more than one athletic drill;
modifying at least one drill in response to a user selection to create a modified workout; and
tracking performance of the modified workout for the user.

4. A computer-readable storage medium comprising computer-executable instructions, which when executed via a processing unit on a computer performs acts, comprising:
tracking an athletic proficiency level of a user of a portable user device;
presenting, on a user interface, a respective sport control affordance for a plurality of sports;
in response to a user selection of one sport control affordance associated with a selected sport, presenting, on the user interface, more than one drill control affordance for a type of athletic drill associated with the selected sport;
in response to a user selection of a particular drill control affordance for a particular type of athletic drill, associating the type of athletic drill with two or more available rhythmic speed settings;
selecting one of the two or more rhythmic speed settings based upon the tracked proficiency level of the user; and
presenting a video depicting one or more of scripted and randomized movement and an audio guide comprising an audio cadence on the portable user device both at the selected rhythmic speed setting that are combined using audio recognition to enhance timing.

5. The computer-readable storage medium of claim 4, further comprising computer-executable instructions, which when executed via the processing unit on the computer performs acts, comprising:
presenting a rhythmic speed adjustment affordance on the user interface;
in response to receiving a user selection of the rhythmic speed adjustment affordance, changing the presented at least one of the video and the audio guide to conform with an adjusted rhythmic speed setting.

6. The computer-readable storage medium of claim 4, further comprising computer-executable instructions, which when executed via the processing unit on the computer performs acts, comprising:
creating a workout comprising more than one athletic drill;
modifying at least one drill in response to a user selection to create a modified workout; and
tracking performance of the modified workout for the user.

7. A portable user device comprising:
a user interface;
a memory containing at least one of video and audio guides; and
a processor coupled to the user interface and the memory and containing a mobility application that when executed by the processor:
tracks in the memory an athletic proficiency level of a user of a portable user device;
presents, on the user interface, a respective sport control affordance for a plurality of sports;
in response to a user selection of one sport control affordance associated with a selected sport, presents, on the user interface, more than one drill control affordance for a type of athletic drill associated with the selected sport;
in response to a user selection of a particular drill control affordance for a particular type of athletic drill, associates the type of athletic drill with two or more available rhythmic speed settings;
selects one of the two or more rhythmic speed settings based upon the tracked proficiency level of the user; and
presents a video depicting one or more of scripted and randomized movement and an audio guide comprising an audio cadence on the portable user device both at the selected rhythmic speed setting that are combined using audio recognition to enhance timing.

8. The portable user device of claim 7, wherein the mobility application that when executed by the processor:
presents a rhythmic speed adjustment affordance on the user interface;
in response to receiving a user selection of the rhythmic speed adjustment affordance, changes the presented at least one of the video and the audio guide to conform with an adjusted rhythmic speed setting.

9. The portable user device of claim 7, wherein the mobility application that when executed by the processor:
creates a workout comprising more than one athletic drill;
modifies at least one drill in response to a user selection to create a modified workout; and
tracks performance of the modified workout for the user.

10. The portable user device of claim 7, further comprising a network interface that couples the processor to the memory for communication via a network.
